(19) United States
(12) Patent Application Publication
(10) Pub. No.: US 2014/0312074 A1
(43) Pub. Date: Oct. 23, 2014

(54) Drive Mechanism for an Injection Device and a Method of Assembling an Injection Device Incorporating Such Drive Mechanism

(71) Applicant: Novo Nordisk A/S, Bagsvaerd (DK)

(72) Inventors: Anders Madsen, Frederiksberg C (DK); Bennie P. Pedersen, Haslev (DK); Carsten S. Andersen, Valby (DK)

(21) Appl. No.: 14/362,288

(22) PCT Filed: Dec. 6, 2012
(86) PCT No.: PCT/EP2012/074683

§ 371 Date: Jun. 2, 2014

Related U.S. Application Data
(60) Provisional application No. 61/568,218, filed on Dec. 8, 2011.

(30) Foreign Application Priority Data
Dec. 6, 2011 (EP) 11192105.2

(51) Int. Cl. F04B 9/02

(52) U.S. Cl. CPC F04B 9/02
USPC 222/333; 29/890.12

(57) ABSTRACT

The present invention relates to a drive mechanism for a spring assisted injection device configured for setting and expelling set doses of a drug. The drive mechanism comprises an assembly provided by a rotatable piston driver (6), a rotatable counter-element (15) and a spring device (19) tensioned between the piston driver (6) and the counter-element (15). During dose setting and during dose expelling the piston driver (6) and the counter-element (15) move relatively along an axis. A releasable interlock (6c, 15d) is provided for maintaining the piston driver (6) and the counter-element (15) in a fixed relative axial condition wherein the spring device (19) is in a tensioned state. The interlock provides for a simplified manufacturing process. The invention also relates to a method of assembling an injection device.

The present invention relates to medical injection devices adapted for injecting apportioned doses of a drug. More specifically, the invention relates to drive mechanisms for medical injection devices incorporating spring assisted dose delivery and methods for manufacture thereof.

In particular, the invention provides improvements with respect to manufacturability and assembling operations during manufacture of a spring assisted injection device.

### BACKGROUND OF THE INVENTION

In the disclosure of the present invention reference is mostly made to the treatment of diabetes by injection or infusion of insulin, however, this is only a preferred use of the present invention.

In order to permit a patient to administer a proper dose of a medicament, various mechanical injection devices have been proposed such as the devices shown in WO 01/95959. Such injection devices facilitate easy and safe dose setting as well as subsequent administration of the set dose by means of a manually operable injection button. Some devices, such as the devices shown in WO 2008/116766 A1, incorporate a spring member wherein energy is stored during a dose setting operation. Upon activation of an injection button, the expelling operation is performed by means of an injection mechanism that utilizes the energy stored in the spring member for expelling the set dose.

WO 2010/033778 A2 discloses a medical injector which includes a spring for urging and displacing a sleeve causing mixing of mixable components of a held reservoir. A releasable retainer retains the sleeve against the force of the spring. Prior to release of the releasable retainer, the releasable retainer cooperates with a channel formed in the body of the injector.

Manufacture of injection devices incorporating a spring assisted injection mechanism usually introduce complexities due to the requirement of assembling one or more springs in a pre-tensioned state. This issue is particularly relevant when manufacturing injection devices that offers user adjustable dose setting.

Having regard to the technical complexity issues of the above-identified prior art, it is an object of the invention to provide a spring assisted injection device that provides for manufacture in a simplified and cost-effective way.

### SUMMARY OF THE INVENTION

In a first aspect the present invention relates to a drive mechanism for an injection device configured for setting and expelling set doses of a drug from a drug-filled cartridge, the drive mechanism comprising:
a piston driver configured for rotation around an axis and for axially driving forward a piston accommodated in a cartridge containing a drug to be expelled,
a rotatable counter-element arranged coaxially with the piston driver, the piston driver and the counter-element being configured for relative axial movement during dose setting from a minimum dose state to a maximum dose state, and
a spring device arranged between the piston driver and the counter-element, the spring device being increasingly tensioned when the dose setting is changed from the minimum dose setting state to the maximum dose setting state, said tension being releasable for driving forward the piston during dose expelling,
   wherein one of the piston driver and the counter-element defines a longitudinal track extending along said axis and the other of the piston driver and the counter-element defines a track follower adapted to engage the longitudinal track, wherein the longitudinal track and the track follower controls the relative rotational position between the piston driver and the counter-element for relative axial positions between the piston driver and the counter-element from the minimum dose setting state to the maximum dose state, and
   wherein a releasable interlock is provided that when activated maintains the piston driver and the counter-element in an interlocked state wherein the relative axial position between the piston driver and the counter-element is fixed and wherein the releasable interlock is so configured that activation and/or release of the interlock requires the piston driver and the counter-element to be positioned relative to each other so that the tension of the spring device exceeds the tension obtained at the maximum dose setting state.

According to the above aspect, by tensioning the spring device and locking the axial movement of the counter-element by means of an interlock between the counter-element and the piston driver, the spring device is in a controlled state, easy to assemble with the remaining components of the device and enabling simple insertion into casing elements forming a housing of the injection device. After the drive mechanism subassembly formed by the piston driver, the counter-element and the spring device has been arranged relative to the housing of the device, the interlock state may be subsequently released allowing the counter-element and the piston driver to axially move relative to each other and enables the dose setting mechanism to be operated between the minimum dose setting state and the maximum dose setting state.

In some embodiments the piston driver comprises a piston driver thread that engages a thread of a further component of the injection device. In accordance with these threaded components, the piston driver rotates and moves in an axial direction as a dose is being dialed up or down.

Also, in some embodiments a piston rod couples movement of the piston driver with movements of the piston during expelling of a set dose. The piston rod and the piston driver may be coupled so as to enable telescopically lengthening of the assembly formed by the piston rod and the piston driver during dose setting.

In some forms the said telescopically lengthening of the piston rod and the piston driver is provided by means of the piston driver thread engaging a thread defined by the piston rod. In other forms the said telescopically lengthening of the piston rod and the piston driver is provided by means of ratchet mechanism, such as an axial ratchet mechanism. The ratchet mechanism may incorporate ratchet teeth providing a one-way lengthening of the assembly formed by the piston driver and the piston rod.

In injection devices where the drive mechanism subassembly is placed radially into the housing of the device, the above assembly may be easily introduced into the housing as the assembly in the interlocked state will fit more easily into the housing. In addition, the radial placement of the subassembly enables better integration with additional components, such as electronic circuitry including position sensors to monitor movements of selected components of the device.

The longitudinal track and the track follower may be formed to unambiguously define the relative rotational position between the piston driver and the counter-element when the piston driver and the counter-element by a relative axial movement is/are shifted between the minimum dose setting state and the maximum dose setting state.

In one form the interlock is so configured that a relative rotational movement between the piston driver and the counter-element is required for activating and/or releasing the interlock.

In further embodiments the interlock may be so configured that activation and/or release of the interlock requires the spring device to be tensioned further than at the maximum dose setting state.

Also, the interlock may be so configured that a force originating from the spring device acts to maintain the interlock in the interlocked state.

In certain embodiments, the longitudinal track is configured for rotationally locking the piston driver relative to the counter-element during relative axial movements from the minimum dose setting state to the maximum dose setting state and vice versa. Hence, the piston driver may be rotationally locked relative to the counter-element to follow rotation of a dose setting knob of the device during dose setting. During dose expelling, the piston driver may remain rotationally locked.

Alternatively, in other embodiments, the longitudinal track may be formed generally longitudinally extending along the axis but with a pitch relative to said axis so that the piston driver and the counter-element rotates relatively to each other when being shifted between the minimum dose setting state and the maximum dose setting state. A corresponding rotation may occur during dose expelling from the set dose to the end of dose position which corresponds to the minimum dose position.

As noted above the piston driver may be configured to move axially relative to the piston rod during dose setting. Also, the piston driver may be configured to move axially relative to the housing during dose expelling.

In some embodiments the piston rod remains stationary relatively to the housing during dose setting.

The track follower may in some embodiments form part of the interlock.

The longitudinal track may define a main direction generally running along said axis. An interlock track may connect with the longitudinal track so that the track follower is receivable in the interlock track. The interlock track may extend sideways, i.e. in a circumferential direction relative to the main direction, so that a relative rotational movement between the piston driver and the counter-element enables activation and/or release of the interlock. Hence during activation and/or release of the interlock the track follower moves along the interlock track.

In some embodiments the interlock track forms a ramp shaped angled surface for releasably maintaining the piston driver and the counter-element in the interlocked state.

In alternative embodiments the piston driver and the counter-element define interlock geometries separate from the track follower and/or the longitudinal track.

In some embodiments, the injection device is for setting and expelling set doses of a drug from a drug-filled cartridge of the kind comprising an outlet and a slideably arranged piston which is driveable in a distal direction to expel the drug through the outlet. The injection device may in some embodiments further comprise a) a housing, b) a piston rod adapted to cooperate with the piston of the cartridge to cause a set dose to be expelled, c) a piston driver coupled to the piston rod, the piston driver being rotated during dose setting away from an initial position to effect the adjustment of the effective length of the piston rod and the piston driver, d) a dosing member mounted rotatably movable but axially fixed in the housing, the dosing member being prevented from rotating during dose setting and allowed to rotate during dose delivery, the dosing member controlling the distal movement of the piston rod during dose injection.

The initial position may correspond to a so-called end of dose state, i.e. the condition that the piston driver assumes after a complete expelling of a previously set dose.

In the present context the term ‘injection device’ should be interpreted to mean a device which is suitable for injecting a drug, such as a liquid drug, into a human or animal body. The injection device is preferably of the kind being suitable for performing repetitive self injection of drug, e.g. insulin for persons having diabetes, or growth hormone. The injection device may be in the form of an injection pen, i.e. of a kind having an elongated shape similar to that of an ordinary pen. Such injection device generally is characterized in that the device part which is intended to rest against an injection site is only held against the skin of the patient during injection of the drug, such as for a duration of less than 1 minute for the complete expelling of a previously set dose.

As mentioned above, the drug is preferably a liquid drug suitable for injection into a human or animal body, e.g. subcutaneously or intravenously. Alternatively, the drug may be a dry drug which has to be reconstituted prior to injection.

The housing may in some embodiments be a part of the injection device which at least substantially encloses the remaining parts of the injection device. Thus, the housing defines an outer boundary of the injection device. The housing may be substantially closed, i.e. it may have substantially solid walls, or it may comprise more or less open parts, such as openings, grids, etc.

The dose setting mechanism is the part of the injection device which is used for setting a desired dose. It may advantageously comprise a part which can be manipulated by an operator and one or more parts which ensure(s) that when an operator manipulates the relevant part, then the injection device is set in such manner that when the injection mechanism is subsequently operated, the desired dose is actually injected by the injection device. In some embodiments, the operator may operate the dose setting mechanism by rotating a rotatable dose knob.

The injection mechanism is the part of the injection device which is used for injecting a desired dose once is has been set by means of the dose setting mechanism. The injection mechanism comprises a piston rod, and the piston rod is adapted to cooperate with a piston positioned in a cartridge. This typically takes place by causing the piston rod to move in an axial direction in the injection device during injection of a previously set dose. The piston rod is typically arranged in the injection device in such a manner that it abuts the piston arranged in the cartridge, and axial movement of the piston rod will therefore cause corresponding axial movement of the piston in the cartridge. Thereby drug is expelled from the cartridge and injected by the injection device. The injection mechanism preferably comprises a part which can be operated by an operator, e.g. an injection button or a release mechanism, e.g. for releasing energy which was previously stored in the spring device during dose setting. The piston driver is axially movable in a proximal direction relatively to the housing during dose setting, and it is axially movable in a distal direction relatively to the housing during injection of a set dose. In the present context the term ‘distal direction’ should generally be interpreted to mean a direction substantially along a longitudinal axis of the injection device, and towards an end being adapted to receive an injection needle. Similarly, in the present context the term ‘proximal direction’ should be interpreted to mean a direction substantially along the longitudinal axis of the injection device, and substantially opposite to the distal direction, i.e. away from the end being adapted to receive an injection needle. The proximal direction is preferably in a direction towards the position of the rotatable dose knob. However, in embodiments incorporating a flexible piston rod that is partly deflected away from an first axis, the remaining parts of the mechanism may be configured for operating along the deflected axis and the included references to distal and proximal directions will generally have to be redefined.

The piston driver is in some embodiments connected to the rotatable dose knob in such a manner that rotating the dose knob causes the piston driver to move axially in a proximal direction. Furthermore, the piston driver is preferably connected to the spring device in such a manner that moving the piston driver axially in a proximal direction causes energy to be stored in the spring device, and in such a manner that releasing energy stored in the spring device causes axial movement of the piston driver in a distal direction. Finally, the piston driver is preferably connected to the piston rod in such a manner that axial movement of the piston driver in a distal direction causes the piston rod to cooperate with the piston to cause a set dose to be delivered.

Retaining means may be arranged to prevent axial movement of the piston driver in a distal direction relatively to the housing during the setting of a dose. In the case that the piston driver is connected to the spring device and the piston rod as described above, the retaining means prevents the spring device from releasing the stored energy and cause the piston rod to cooperate with the piston to inject drug during dose setting. Thus, it is prevented that drug is accidentally spilled, and it is ensured that a correct dose is being set. Controlling this by axially retaining the piston driver rather than locking the piston rod directly has the following advantage. When a cartridge is empty and therefore has to be replaced, it is necessary to return the piston rod to an initial position corresponding to a full cartridge. In the case that axial movement of the piston rod in a distal direction during dose setting is prevented by directly locking the piston rod, e.g. by means of a locking item or a dosing member, it may be difficult to return the piston rod during replacement of the cartridge. This is particularly the case when the piston rod and the locking item/dosing member are engaged in such a manner that they tend to jam. However, according to the present invention axial movement of the piston rod in a distal direction is prevented by axially retaining the piston driver, and the risk of jamming the piston rod during replacement of the cartridge is thereby minimised, since the piston rod is allowed to return freely to the initial position.

The retaining means may be a dosing member being axially fixed relatively to the housing, and the dosing member may be adapted to be rotationally locked relatively to the housing during dose setting, and it may be adapted to be able to perform rotational movement relatively to the housing during injection of a set dose. According to this embodiment, when the dosing member is rotationally locked relatively to the housing, it axially retains the piston driver, i.e. it prevents the piston driver from performing axial movements in a distal direction. However, when the dosing member is allowed to perform rotational movement relatively to the housing it allows the piston driver to move axially in a distal direction.

The dosing member and the piston driver may be connected via mating threads formed on the piston driver and the dosing member, respectively. According to this embodiment the piston driver can be moved axially in a proximal direction by rotating the piston driver, thereby allowing it to climb the threaded connection between the dosing member and the piston driver. However, the threaded connection prevents that the piston driver is pushed in a purely axial movement in a distal direction as long as the dosing member is not allowed to rotate relatively to the housing. When the dosing member is subsequently allowed to rotate, the piston driver is allowed to move axially in a distal direction while causing the dosing member to rotate.

The injection device may further comprise a locking item being movable between a locking position in which it prevents the dosing member from rotating relatively to the housing, and an unlocking position in which the dosing member is allowed to rotate relatively to the housing. According to this embodiment the locking item is in its locking position during dose setting and in its unlocking position during injection of a set dose. Mating teeth may be formed on the dosing member and the locking item, respectively, and these mating teeth may engage when the locking item is in the locking position. When the locking item is moved into its unlocking position, the mating teeth are, in this case, moved out of engagement, thereby allowing mutual rotational movement between the dosing member and the locking item.

The locking item may be moved from the locking position to the unlocking position in response to operation of the injection mechanism. According to this embodiment, the locking item is automatically moved into the unlocking position when a user operates the injection mechanism. Thereby the injection device is automatically shifted from a state where a dose can be set into a state where a dose can be injected when the user operates the injection mechanism. Thereby the user only has to perform a single operation in order to cause a set dose to be injected, and the injection device is thereby very easy to operate.

As an alternative to a dosing member, the retaining means may, e.g., be or comprise a key and groove connection, one or more braking elements, one or more slidable locking elements, and/or any other means being suitable for axially retaining the piston driver as described above during dose setting.

The piston driver may be prevented from performing rotational movements relatively to the housing during injection of a set dose. According to this embodiment the piston driver moves in a purely axial manner relatively to the housing during injection of a set dose. This provides a very simple movement pattern, and the risk that the injection device jams during injection of a set dose is minimised.

The piston driver and the piston rod may be connected via mating threads formed on the piston driver and the piston rod, respectively. According to this embodiment, the piston driver is preferably moved along this threaded connection during dose setting. During injection the piston rod is preferably moved along the piston driver in an axial movement.

In a preferred embodiment the piston driver is threadedly connected to the piston rod as well as to a dosing member. For instance, the piston driver may comprise an inner thread arranged to engage an outer thread of the piston rod and an outer thread arranged to engage an inner thread of the dosing member. According to this embodiment, the piston rod, the piston driver and the dosing member may be arranged relatively to each other in such a manner that at least part of the piston driver surrounds at least part of the piston rod, and at least part of the dosing member surrounds at least part of the piston driver. As an alternative, the piston rod may be hollow, and the piston driver may, in this case comprise an outer thread arranged to engage an inner thread of the hollow piston rod.

The injection device may further comprise means for preventing rotational movement of the piston rod during dose setting. The means for preventing rotational movement of the piston rod may comprise a key and groove connection between the piston rod and a member being fixed relatively to the housing. Such a key and groove connection prevents the piston rod from rotating relatively to the housing, but relative axial movement is possible. The member is fixed relatively to the housing during normal operation, i.e. at least when a cartridge is inserted in the housing. However, the member may advantageously be fixed to the housing in such a manner that it is released, e.g. allowing rotational movements of the member relatively to the housing, during change of cartridge. Such an arrangement would allow the piston rod to be moved back during change of cartridge. This will be explained in more detail below with reference to the drawings.

Alternatively, the means for preventing rotational movement of the piston rod may comprise a third thread connection provided between the piston rod and a member being fixed relatively to the housing. The remarks set forth above relating to the member being fixed to the housing are equally applicable here. The third thread connection preferably has a pitch being directed in a direction which is opposite to the direction of the first thread. According to this embodiment the first thread connection between the dosing member and the piston rod and the third thread connection between the member and the piston rod in combination prevent rotational movement of the piston rod during dose setting, and thereby prevent axial movement of the piston rod during dose setting.

The piston driver may be connected to the dose knob via a key and groove connection. In this case the piston driver is simply rotated along with the dose knob during dose setting, and the dose knob and the piston driver may be allowed to perform mutual axial movements.

The operation of the dose setting mechanism causes energy to be stored in a spring device, and the injection mechanism is driven by releasing energy previously stored in said spring device during dose setting. The spring device may, e.g., comprise a spring, such as a compressible spring, an extension spring or a torsion spring, or it may be or comprise any other suitable means capable of storing mechanical energy and subsequently releasing the stored energy. Such an injection device is very easy to use for persons having poor dexterity or low finger strength, e.g. elderly people or children, because only a relatively small force needs to be applied by the user in order to inject a set dose, since the necessary mechanical work is carried out by the spring device. Furthermore, in injection devices where the injection is performed by releasing energy previously stored in a spring device, the piston rod is normally moved during injection by applying a pushing force to the piston rod in a substantially axial direction.

The injection device may further define a release mechanism for releasing energy stored in the spring device, thereby causing a set dose to be injected. The release mechanism may, e.g., comprise a release button which the user operates. The release mechanism is preferably axially movable, and it may be operable by a user pressing a release button in a substantially axial direction. In this case the release button may be integral with the dose knob.

In a second aspect, the invention relates to a method of assembling an injection device incorporating a drive mechanism in any of the forms as described above.

Such method may comprise the steps of:

a) providing the spring device, the piston driver and the counter-element,

b) forming a drive mechanism subassembly by arranging the piston driver, the spring device and the counter-element relative to each other and operating the piston driver and the counter-element relatively to each other so that the spring device is tensioned,

c) moving the piston driver and the counter-element relatively to each other into a state where the tension of the spring device exceeds the tension of the spring device obtained when in the maximum dose setting state,

d) activating the releasable interlock for maintaining the piston driver and the counter-element in an interlocked state wherein the relative axial position between the piston driver and the counter-element is fixed and wherein the spring device is in a tensioned state.

In accordance herewith, the releasable interlock is activated only when the piston driver and the counter-element are positioned relatively to each other to assume a state where the tension of the spring device exceeds the tension of the spring device obtained when in the maximum dose setting state. Hence, the interlock mechanism cannot interfere with the operation of the dose setting mechanism during subsequent operations, e.g during use of the assembled injection device such as during dose setting operations and during dose expelling operations.

The assembling method may further comprise the step of providing a housing, and, subsequent to step c): the steps of,

e) positioning the assembly relative to the housing, and

f) releasing the interlock thereby enabling the piston driver and the counter-element to be axially moveable relative to each other between the minimum dose setting state and the maximum dose setting state.

Further, the method of assembling the injection device as defined above may further comprise that in step a), i.e. providing the spring device, the piston driver and the counter-element, these elements may be provided such that:
one of the piston driver and the counter-element defines a longitudinal track extending along said axis, and
the other of the piston driver and the counter-element defines a track follower adapted to engage the longitudinal track, wherein the longitudinal track and the track follower controls the relative rotational position between the piston driver and the counter-element for relative axial positions between the piston driver and the counter-element between the minimum dose setting state and the maximum dose state, and
   wherein an interlock track connects with the longitudinal track so that the track follower is receivable in the interlock track, the interlock track extending substantially in a circumferential direction sideways relative to longitudinal track so that activation and/or release of the interlock requires a relative rotational movement between the piston driver and the counter-element forcing the track follower along the interlock track.

The assembling method may further comprise the step, subsequent to step e) of:

g) operating the piston driver and the counter-element relative to each other for setting the dose between the minimum dose setting state and the maximum dose setting state or vice versa.

As noted above, any of the details, embodiments and forms described in connection with the first aspect may be used in the assembling method according to the second aspect.

### BRIEF DESCRIPTION OF THE DRAWINGS

The invention will now be described in further detail with reference to the accompanying drawings in which:

FIG. 1a shows a shows cross sectional side view and FIG. 1b a top view of mechanical parts of an injection device suitable for being manufactured by a method according to the present invention, the injection device being in a position where it is ready to set a dose,

FIGS. 2a and 2b shows similar views of the injection device of FIG. 1 in a position where a dose has been set,

FIGS. 3a and 3b shows similar views of the injection device of FIGS. 1 and 2 in a position where a dose has been set and the injection button has been pushed,

FIGS. 4a and 4b shows similar views of the injection device of FIGS. 1-3 in a position where a dose has been injected and the injection button is still pushed,

FIG. 5 is an exploded view of selected parts of the injection device of FIGS. 1-4,

FIG. 6 is a perspective view of device similar to the device shown in FIGS. 1-5, including a first subset of sensor elements of the electronic sensing system according to the present invention,

FIG. 7 is a perspective view similar to FIG. 6 and including first and second subsets of sensor elements of the electronic sensing system,

FIG. 8 is a perspective view similar to correspond to FIG. 7 and further showing a switch frame,

FIG. 9 is a top view of components shown in FIG. 6 and including a housing component,

FIG. 10 is a top view corresponding to FIG. 7,

FIG. 11a shows a schematic representation of a sensor system associated with a piston driver in the form of a dosage tube,

FIG. 11 b shows a schematic representation of a sensor system associated with a locking nut,

FIGS. 12a and 12b represent tables of sensor values of the sensor systems of FIG. 11a and FIG. 11 b respectively,

FIG. 13 shows a detailed view of an embodiment of a locking nut comprising a Gray code pattern for use in an injection device,

FIG. 14 is a schematic view of the metal layers disposed on the locking nut shown in FIG. 13,

FIGS. 15a, 15b and 15c show side views of selected components of the drive mechanism according to the invention during manufacture respectively representing a first, a second and third assembly state, and

FIGS. 16a, 16b and 16c show cross sectional side views of the drive mechanism during manufacture respectively representing a fourth, a fifth and a sixth assembly state.

### DETAILED DESCRIPTION OF THE DRAWINGS

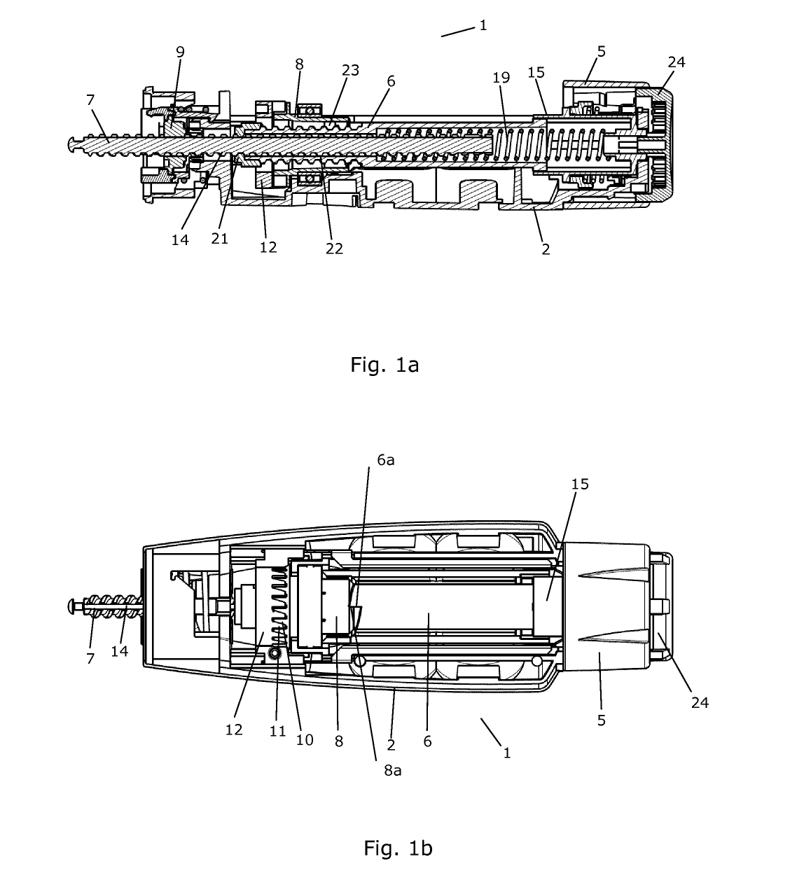

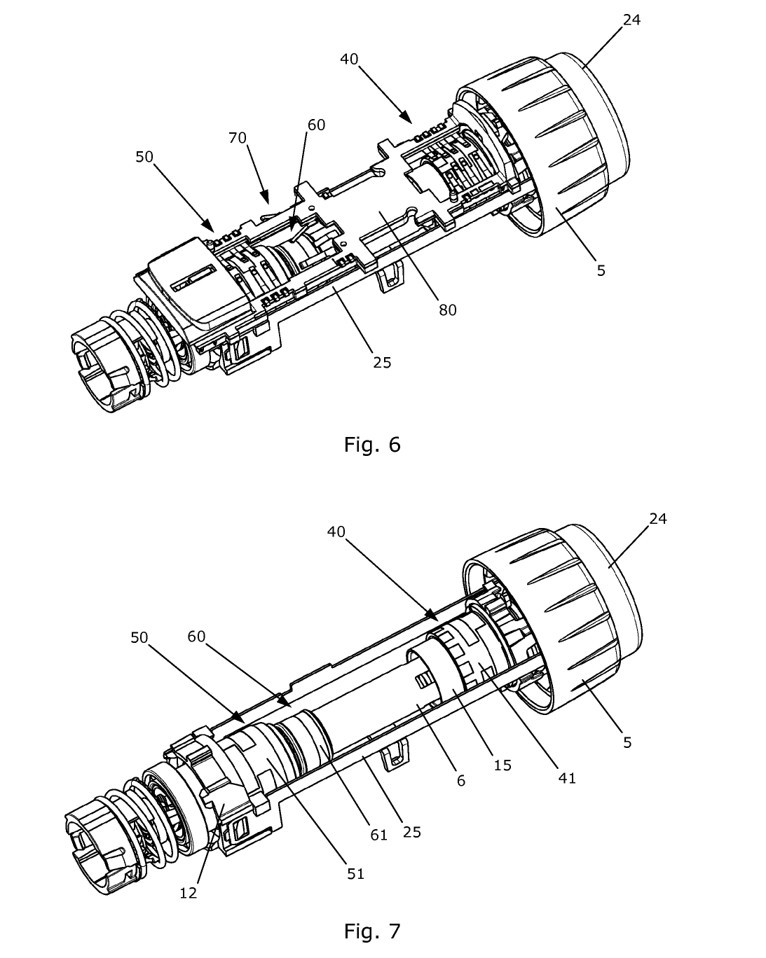

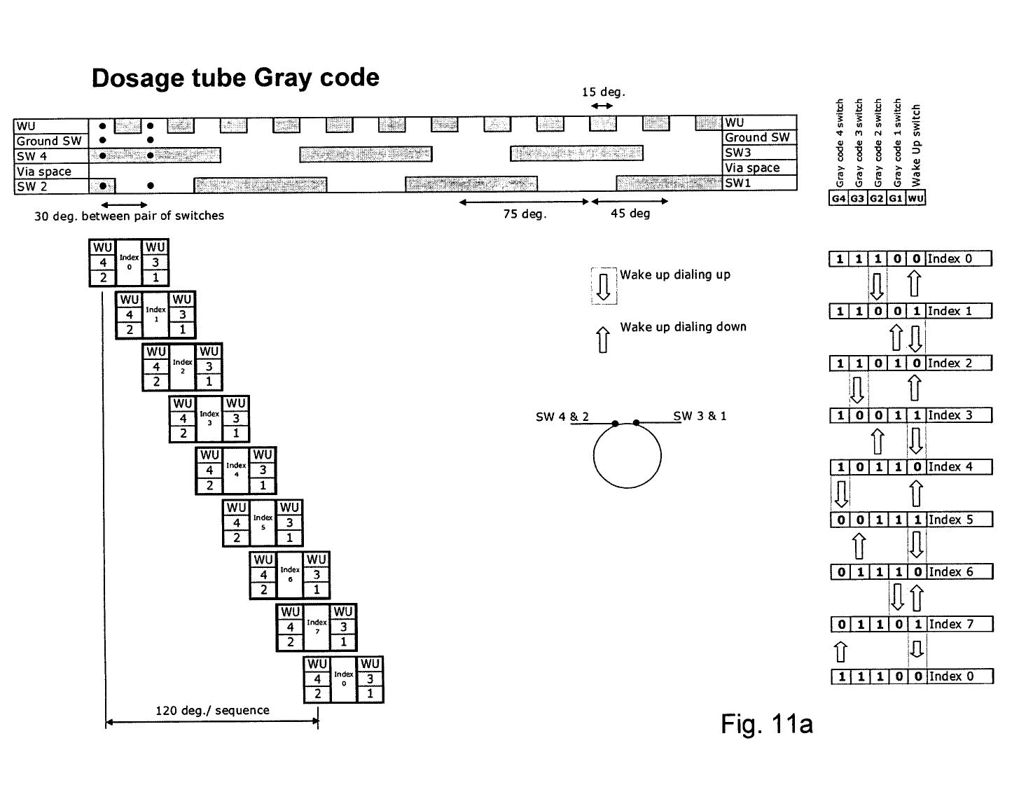

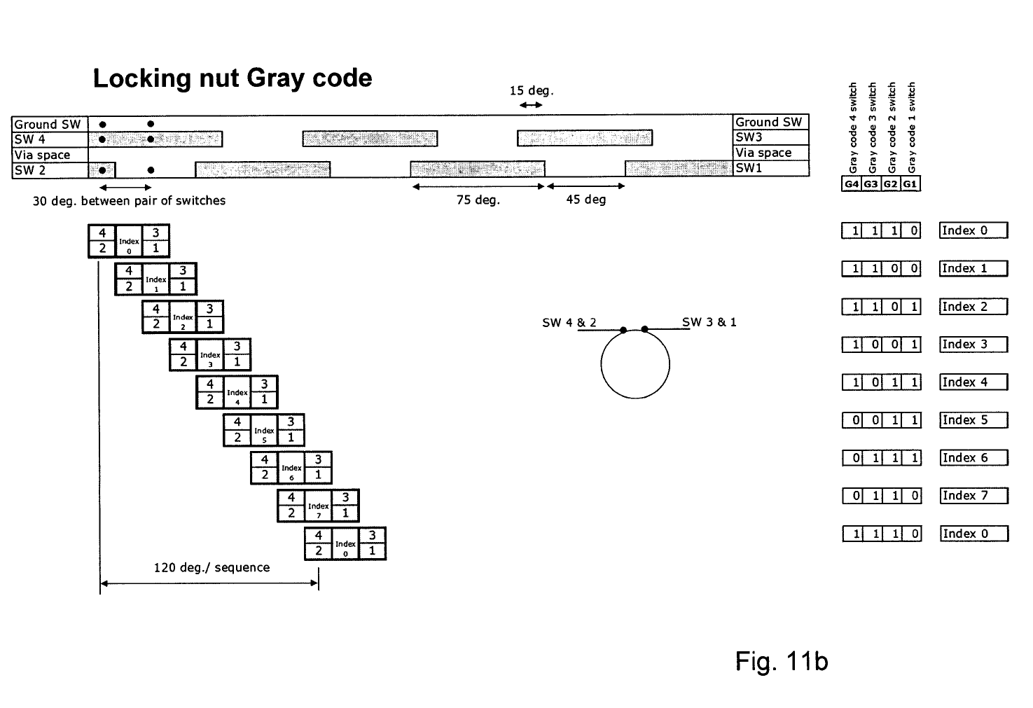

FIGS. 1 through 5 illustrate an injection device 1 comprising a drive mechanism, i.e. a dose setting mechanism for setting a dose of a drug and a dose injection mechanism for injecting previously set doses. Such drive mechanism is suitable for use with a sensing system described in connection with FIGS. 6 through 12. The device shown in FIGS. 1-5 generally corresponds to the embodiment shown in FIGS. 11-15 of WO 2008/116766 A1, this document being incorporated herein by reference.

The drive mechanism included in injection device 1 is adapted to operate in two mechanical operational modes, respectively designated Dose Setting Mode and Dosing Mode. In Dose Setting Mode, dose setting may be performed by dialing up and down a manually operable dose setting member. In this mode, the piston rod of the device is held stationary so that no dose will be expelled. In Dosing Mode, altering an already set dose is prevented while the expelling of an already set dose can be performed. The mechanism may include a mechanical transition zone between the Dose Setting Mode and the Dosing Mode, the transition zone being designated Safe Mode. Safe Mode is a zone ensuring that neither dose setting nor dose expelling can be performed.

In FIG. 1, the injection device 1 is shown in a position where it is ready for setting a dose. In FIG. 1a the injection device 1 is shown in a cross sectional side view. In FIG. 1 b, the injection device 1 is shown in a top view. For reasons of clarity some of the parts of the injection device 1 have been omitted in the drawings in order to show parts arranged in the interior of the injection device and to better illustrate their operation.

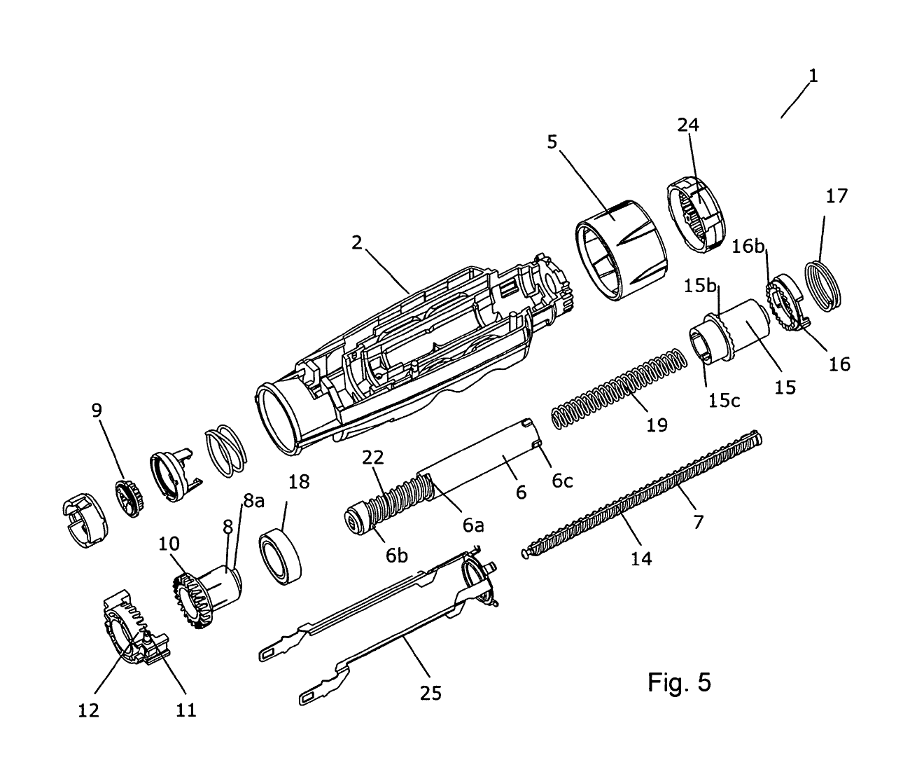

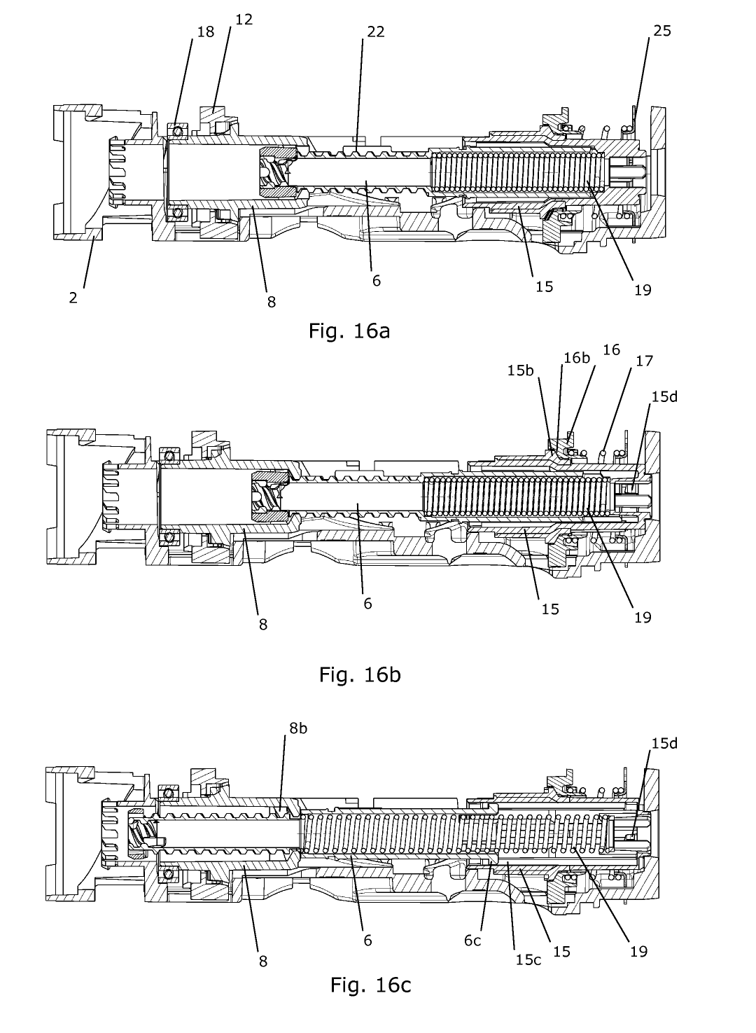

The injection device 1 of FIG. 1 comprises a piston driver 6 in the form of a dosage tube and a dosing member which in the following will be referred to as a locking nut 8. The device 1 further comprises a piston rod 7. In this embodiment the piston driver 6 is threadedly connected to the piston rod 7 via an inner thread 21 formed on the piston driver 6 and a corresponding outer thread 14 formed on the piston rod 7. The piston driver 6 is further provided with an outer thread 22. The piston driver 6 and the locking nut 8 are threadedly connected via the outer thread 22 of the piston driver 6 and inner thread 23 formed on the locking nut 8. The outer thread 22 of the piston driver 6 covers only part of the length of the piston driver 6. Thereby the distance which the piston driver 6 is allowed to travel relatively to the locking nut 8 is limited, and the ends of the outer thread 22 of the piston driver 6 define end positions of the relative movement between the piston driver 6 and the locking nut 8. Accordingly, it is not possible to set a dose which is smaller than a dose corresponding to one end position, and it is not possible to set a dose which is larger than a dose corresponding to the other end position. The minimum dose setting limit may be defined by rotational stop surfaces 6a and 8a respectively being formed by piston driver 6 and locking nut 8. In the shown embodiment, a corresponding rotational stop (not visible in FIGS. 1-4) is associated with the piston driver 6 and locking nut 8 for defining the maximum allowable dose setting (cf. FIGS. 5 and 16c). A set of teeth 10 formed on the locking nut 8 and a set of teeth 11 formed on the locking item 12 engage as can be seen in FIG. 1 b. The locking item 12 is rotationally locked to the housing component 2, and the engagement of the teeth 10, 11 thereby prevents the locking nut 8 from rotating. By means of the teeth 10,11 the locking nut 8 is designed to be locked rotationally relative to the housing 2 in a number of pre-defined rest-positions.

In the injection device 1, the dose setting member forms a dose knob 5. When it is desired to set a dose the dose knob 5 is rotated. The dose knob 5 is rotationally locked to injection button 24 via a first spline connection. The injection button 24 is rotationally locked to a dose setting item via a second spline connection. In the following the dose setting item will be referred to as a counter-element 15. In the shown embodiment, the counter-element 15 is rotationally locked to the piston driver 6 via a third spline connection. Accordingly, when the dose knob 5 is rotated, the piston driver 6 is rotated along. Due to the threaded connection between the piston driver 6 and the locking nut 8, and because the locking nut 8 is prevented from rotating, due to the engagement between teeth 10, 11, the piston driver 6 is thereby moved axially in a proximal direction relative to the locking nut 8, and in a spiraling movement. Simultaneously, the piston driver 6 climbs along the piston rod 7 which remains fixed relative to the housing component 2.

In the shown embodiment, the injection device includes a spring device in the form of a helical compression spring 19 arranged internally between the piston driver 6 and the counter-element 15. During dose setting, the axial movement of the piston driver 6 causes compressible spring 19 to be compressed, i.e. energy is stored in the compressible spring 19. The distance traveled by the piston driver 6 corresponds to the dose being set.

An initially set dose may be dialed down fully or partly by reversing the direction of rotation of dose knob 5. Such dialing down may be performed all the way to the zero dose dial position to thereby return the piston driver 6 to the initial relative rotational position between the piston driver 6 and the locking nut 8. The injection device 1 may include an indexing mechanism whereby the dose knob 5 is configured to move in discrete rotational steps corresponding to the desired dose increments, i.e. providing a number of pre-defined rest-positions which may correspond to the number of locking positions between locking nut 8 relative to the housing 2. Referring to FIG. 5, such an indexing mechanism may be provided as a spring biased click-mechanism including a knurled ring surface 15b on counter-element 15 which engages a corresponding knurled surface 16b on a ring shaped surface defined by indexing member 16. Click spring 17 provides a biasing force for biasing the knurled ring surface on counter element 15 against the corresponding knurled surface on ring-shaped indexing member 16. In the shown embodiment, the dose knob 5 is adapted to rotate in 24 rotational steps during each revolution that the dose knob 5 undergoes during dose setting, i.e. corresponding to 24 dose increments. The minimum and maximum limit stops defined between piston driver 6 and locking nut 8 are decisive for the relative rotational and axial movement between these components and may be defined to a total of say 80 or 100 dose increments.

In some embodiments, the force originating from the compressible spring 19, when compressed, may tend to automatically dial down an initially set dose. However, the inclusion of an indexing mechanism may prevent this by adequately designing the indexing mechanism to provide reluctance against self-returning of the dose knob 5.

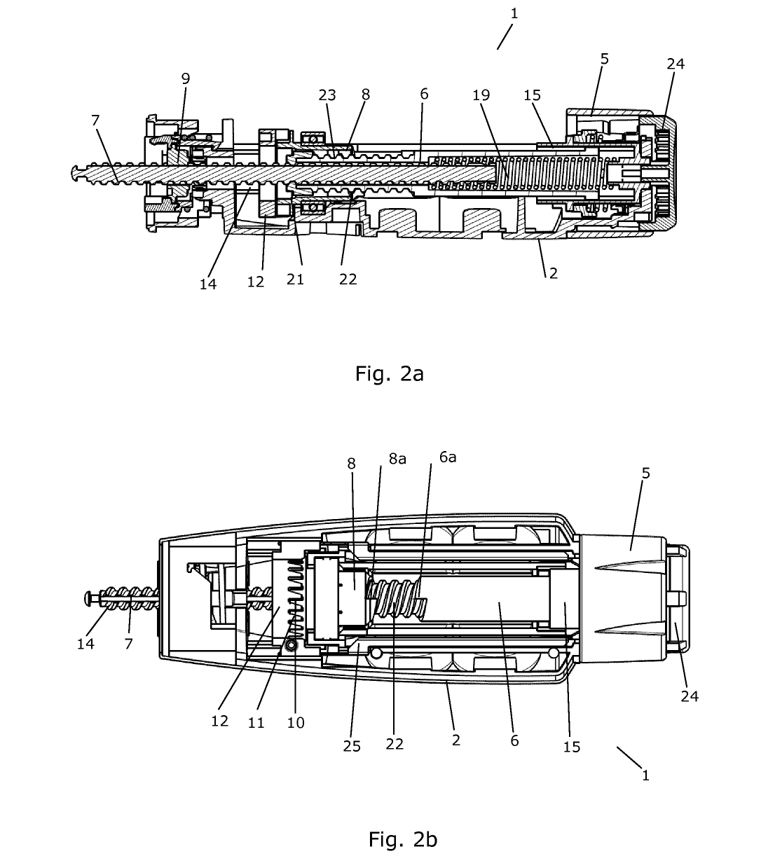

FIGS. 2a and 2b show the injection device 1 of FIGS. 1a and 1b in a position where a dose has been set. In FIG. 2a the injection device 1 is shown in a cross sectional view, and in FIG. 2b the injection device 1 is shown in a top view with some of the parts omitted for the sake of clarity, similar to FIG. 1 b.

Comparing FIGS. 1a+1b and FIGS. 2a+2b it is clear that the piston driver 6 has been moved in a proximal direction and that the compressible spring 19 has been compressed. In FIG. 2a it can be seen that the piston driver 6 is arranged in such a manner that the inner thread 23 of the locking nut 8 is positioned very close to one of the ends of the outer thread 22 of the piston driver 6. Thus, the dose which has been set is very close to the maximum settable dose. In FIG. 2b the outer thread 22 of the piston driver 6 is visible.

In FIG. 2b it can be seen that the teeth 10 formed on the locking nut 8 and the teeth 11 formed on the locking item 12 are still engaged, i.e. the locking nut 8 is still prevented from rotating relatively to the hosing 2. Thus, the piston driver 6 is retained in the position shown in FIG. 2.

When it is desired to inject the set dose, the injection button 24 is pushed in a distal direction, i.e. towards the housing component 2. The injection button 24 is connected to the locking item 12 via connecting part 25. Accordingly, pushing the injection button 24 causes the locking item 12 to move along in a distal direction, thereby moving the teeth 10, 11 out of engagement, allowing the locking nut 8 to rotate. The injection button 24 is configured in such a manner that it automatically returns to its initial distal position when external pressure acting on the injection button 24 is released. In the shown embodiment this is obtained by means of click spring 17.

The locking nut 8 may be mounted relative to the housing by means of a ball bearing or similar to provide a low-frictional rotation of the locking nut 8 during dosing.

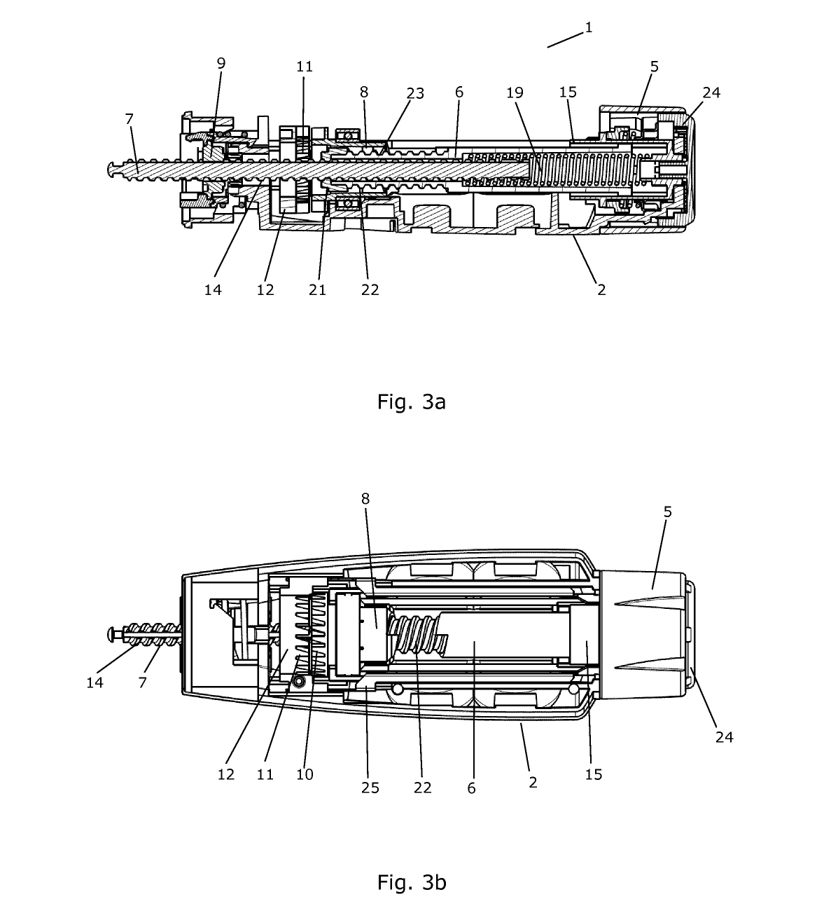

FIGS. 3a and 3b show the injection device 1 of FIGS. 1 and 2 in a position where the injection button 24 has been pushed in a distal direction as described above. In FIG. 3b it can be seen that the teeth 10, 11 have been moved out of engagement. The position of the piston driver 6 is the same as in FIG. 2, i.e. the injection device 1 has not yet started injecting the set dose.

The compressed spring 19 pushes against the piston driver 6, thereby urging it in a distal direction. Since the locking nut 8 is now allowed to rotate, the piston driver 6 is allowed to move in a distal direction, while forcing the locking nut 8 to rotate due to the connection between the outer thread 22 of the piston driver 6 and the inner thread 23 of the locking nut 8. The energy stored in the compressed spring 19 will cause the piston driver 6 to perform this movement. Due to the connection between the inner thread 21 of the piston driver 6 and the outer thread 14 of the piston rod 7, the piston rod 7 is moved along in this movement. In use of the injection device 1, the piston rod 7 is arranged in abutment with a piston (not shown) arranged in a cartridge. Accordingly, moving the piston rod 7 as described above causes the set dose of drug to be expelled from the injection device 1. The injection movement may be halted at any time during injection by releasing the injection button 24. The dose movement may be continued by once again pushing the injection button 24 in the distal direction.

In the shown embodiment, the injection button 24 is provided with a plurality of axially extending teeth (not referenced) arranged to releasably engage corresponding teeth (not referenced) formed in the housing component 2 (cf. FIGS. 2a, 3a and 9). The engagement of the two sets of teeth is initiated upon pressing in of the injection button 24, and the engagement is released when the injection button 24 moves into its proximal position. Hence, manipulation of the dose knob 5 to alter a set dose during the injection movement is prevented.

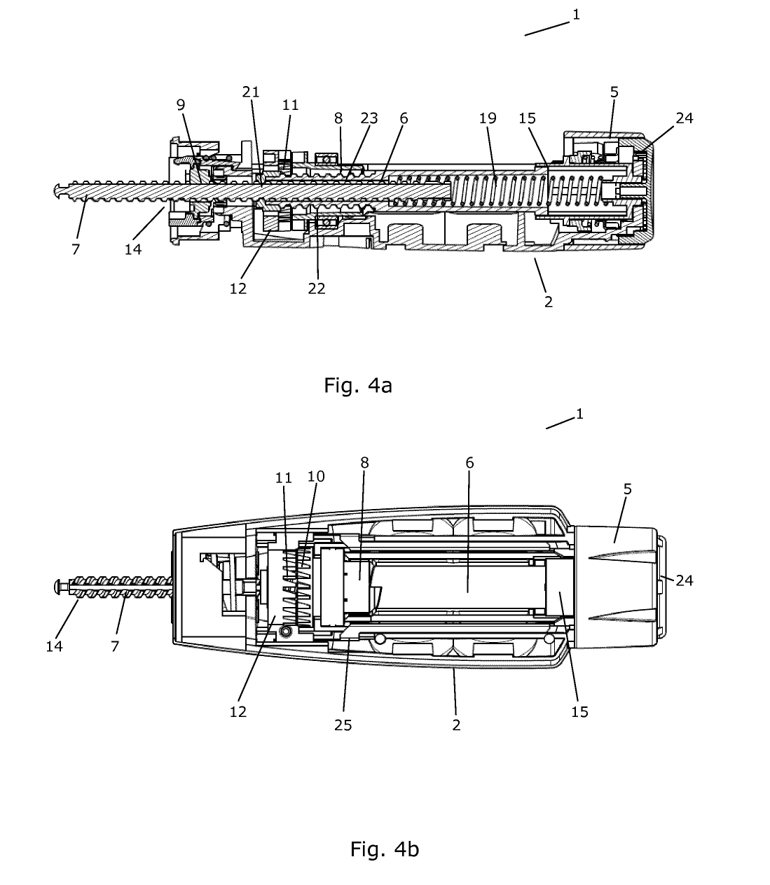

FIGS. 4a and 4b show the injection device 1 of FIGS. 1-3 in a position where injection of the set dose has been completed. Comparing FIG. 3 and FIG. 4 it can be seen that the piston driver 6 has been returned to the position shown in FIG. 1. However, the piston rod 7 has been moved in a distal direction as compared to the position shown in FIG. 1, thereby indicating that a dose has been injected.

In accordance with the above, as the locking nut 8 only rotates during the injection process, i.e. from the start of the dosing movement of piston driver 6 till the end of dose state is reached, the locking nut 8 performs as a dosing member for metering doses expelled from the device.

In the shown embodiment, the piston rod 7 is rotationally locked with respect to the housing component 2 during dose setting and injection operations. However, in an alternative embodiment, the piston rod 7 may be configured to rotate during the dosing movement in a manner as described in WO 2006/114395. As known in the art, the rotational lock or the rotational guiding of piston rod 7 relative to housing component 2 may be provided by means of a locking disc 9 which engages a track or thread on piston rod 7 and which is locked relative to the housing during the dose setting and dose injection process.

FIG. 5 is an exploded view of the injection device 1 of FIGS. 1-4. For the sake of clarity, only the parts necessary for explaining the operation of the injection device 1 are shown. In FIG. 5 the connecting part 25, the knurled disc 16, the click spring 17 and the ball bearing 18 are clearly visible.

Turning now to FIGS. 6 through 10 a dose setting and injection mechanism is shown which in most aspects are similar to the one of the device shown in FIGS. 1-5 but which include electronic components enabling the position detection of specific mechanical components incorporated in the device and allowing the monitoring of the mechanical components during operation of the device 1. Further, the electronic components may include an electronically controlled display and/or communication means for utilizing information relating to the detected position data, e.g. a number of set and/or expelled doses. In FIGS. 6-10, the parts that are shown which correspond to similar parts shown in FIGS. 1-5 have been provided with identical reference numerals. Likewise, only the parts necessary for explaining the operation of the electric components of the injection device 1 are shown.

In an exemplary embodiment and as identified in FIG. 6, four switch arrangements are provided for detecting the individual mechanical movements and states within the device mechanism. A first sensor arrangement 40 is associated with the piston driver 6 to provide positional data relating to the rotational position of the piston driver 6 relative to the device housing. A second sensor arrangement 50 is associated with the locking nut 8 to provide positional data relating to the rotational position of the locking nut 8 relative to the device housing. A third sensor arrangement 60 is also associated with the piston driver 6 and provides information relating to the axial position of piston driver 6, i.e. whether the piston driver 6 is within a predefined amount of axial travel distance from the end of dose position. Further, a fourth sensor arrangement 70 may include a switch which provides data relating to the axial position of the injection button 24, thus also the axial position of connecting part 25 and locking item 12. Hence, sensor arrangement 70 provides data as to whether the injection device 1 is in the Dose Setting Mode or in the Dosing Mode as defined above.

In the shown embodiment, the sensor arrangements 40, 50, 60 and 70 are formed as conductive switch based sensors which are coupled to an electronic control circuit incorporating a processor and being powered by a power source. In FIG. 6, a switch frame 80 is visible which is configured to hold and retain various contact elements in the form of contact arms of the sensor arrangements 40, 50, 60 and 70 in fixed relationship with the housing component 2.

The first sensor arrangement 40 used for detecting a set dose is based on a principle of detecting the rotational motion between the piston driver 6 and the switch frame 80. As the counter-element 15 rotates together with the piston driver 6 and as the counter-element 15 is mounted axially fixed in the device 1, the counter-element 15 is utilized for detecting rotational movements during a dose setting operation. By keeping track of the rotation of counter-element 15 it is possible to determine the dose set. The sensor arrangement 40 is implemented as a Gray code pattern (referenced first Gray code pattern 41) which is fixedly arranged relative to counter-element 15. The first Gray code pattern 41 is formed as a cylindrical drum being swept by a set of contact arms comprised within the switch frame 80 as the piston driver 6 is rotated. Hence, it is possible to detect direction and keep count of the net dose set. The set of contact arms are formed as a group of eight contact arms below referred to as the first group of contact arms 42.

The second sensor arrangement 50 used for detecting the amount dosed is based on the same principle utilizing a first Gray code pattern 51 provided as a cylindrical drum fixedly arranged relative to the locking nut 8. This Gray code pattern 51 is being swept by a second group of contact arms 52 which in the shown embodiment consist of six contact arms.

The first and second gray code patterns 41 and 51 are provided as galvanically conducting patterns having a series of electrically insulating fields disposed thereon. Alternatively, the first and second Gray code patterns may be formed as a generally electrically insulating base material having a plurality of galvanically conducting fields disposed thereon. In the shown embodiment the code patterns 41 and 51 are provided as metallic or metallized sleeves which are fixedly attached to counter-element 15 respectively to locking nut 8.

In alternative embodiments, the first Gray code pattern 41 and/or the second Gray code pattern 51 may be provided as unitarily formed into counter-element 15 respectively to locking nut 8, such as being fabricated using MID technology (Molded Interconnect Devices). Typical known methods for producing conductor tracks on three-dimensional products include, for example, two-component injection molding, hot-stamping, mask-exposure methods and thin-film insert molding.

In a particular embodiment, the first Gray code pattern 41 and/or the second Gray code pattern 51 are formed by Laser Direct Structuring (LDS) whereby the counter-element 15 and/or the locking nut 8 are formed by an initially non-conductive doted thermoplastic material. The thermoplastic material on which the conductive areas are to be formed are activated by means of targeted laser radiation and then metallized in a chemical bath. Typically, the LDS process involves forming a first copper layer on the activated areas by means of a chemical metal-deposition process in a current-free copper bath, then a chemical nickel layer is applied electroless on top of the copper layer and finally a flash gold layer is applied electroless on top of the nickel layer to provide a corrosion resistant surface.

FIG. 13 shows the locking nut 8 which has been provided as an injection-molded component being formed by a non-conductive thermoplastic support material having at least the surface portion intended for holding the Gray code pattern 51 compounded with a radiation activatable metal complex. The molded component includes surface geometries 10 described above which is configured to cooperate with corresponding surface geometries 11 defined by locking item 12. FIG. 13 further shows the Gray code pattern 51 comprising electrically non-conductive areas and conductive areas. However, in contrast with the above described electroless application of layers, in order to provide a superior wear resistance as well as an effective electrical conductivity an outer layer of hard gold is formed on top of the previously formed metallic layers of the laser activated areas. The hard gold (around 99.7% pure) is made hard during the plating process by adding cobalt and/or nickel at levels of approximately 0.1% to 0.3%. The hard gold layer is applied by a galvanical process.

In this embodiment, and as indicated in FIG. 14, the laser activated areas are firstly deposed electroless with copper in a layer thickness in the range of approximately 5 to 8 μm. Then a copper layer is deposed galvanically on top of the electroless copper layer, the galvanic copper layer having a thickness of approximately 20 to 25 μm. Subsequently, a layer of nickel is deposed galvanically on top of the galvanic copper layer, the nickel layer having a thickness of approximately 3 to 5 μm. Finally the hard gold layer is applied galvanically with a layer thickness of 0.25 to 2.5 μm, preferably in the range of 1.25 to 2.5 μm.

The galvanic copper layer is optional and provides for levelling the finished product to obtain a particular smooth surface of the conductive areas of the Gray code pattern. In other embodiments, this layer may be omitted.

The counter-element 15/Gray code 41 may be manufactured by a similar process as described above in connection with the locking nut 8/Gray code pattern 51, including the injection-molding of a thermoplastic support material to provide the surface geometries 15b to cooperate with corresponding surface geometries 16b of knurled disc 16 (see FIG. 5).

By the above process a particular durable surface for the Gray code pattern is achieved which, especially for switch devices having contact elements wiping the surface of the code surface (containing the non-conductive and conductive areas), provides superior wear resistance, durability and electrical conductivity. Hence, in the particular application for the locking nut 8 above and/or for the counter-element 15, a particular reliable encoder solution is provided.

As for the contact elements, the state of each individual contact arm is detected by the switch sensor interface of the electronic control circuit and the information is processed by an algorithm implemented in the switch sensor interface. In this way the switch sensor interface counts the amount set, counts the amount dosed, and presents the value of these counters to the processor for further processing the data.

Electrically the sensors are configured as switches connected to ground, and the corresponding inputs to the electronic control circuit are held high by pull-up resistors to ensure a well-defined signal level. An open switch will not consume any power, but a closed switch will consume power as its pull-up resistor connects supply voltage and ground. A power conservation strategy is implemented that disables the pull-up resistors for the switches that are closed in the same manner as described in WO 2010/052275.

Such sensor system will not consume power continuously, but with this strategy only transitions that results in switches being closed can be detected. A switch that opens will not generate a rising voltage on its corresponding input since the pull-up resistor for that input has been disabled.

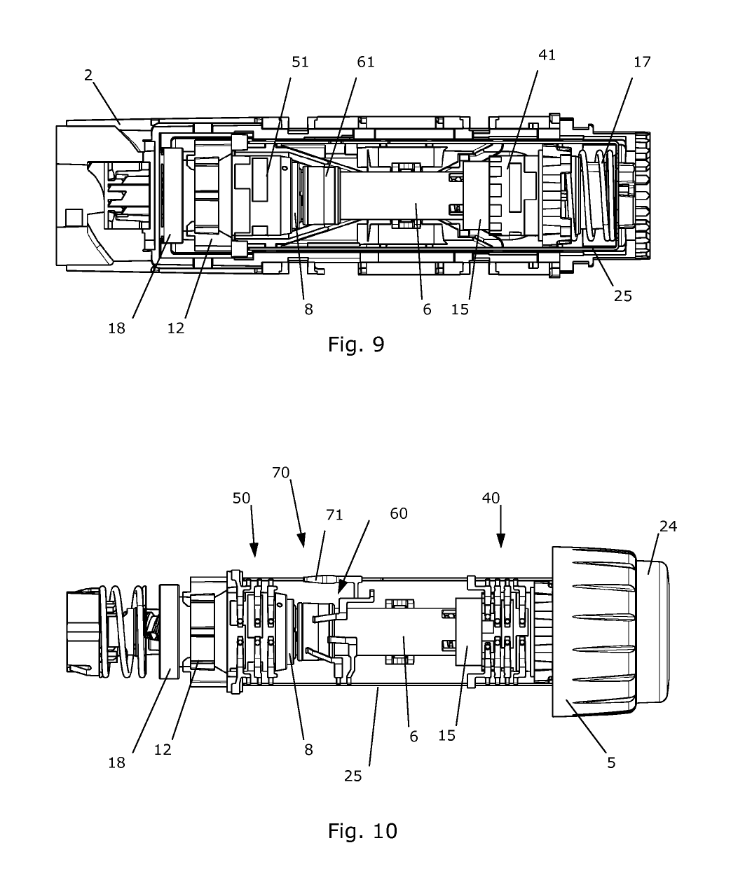

FIG. 7 and FIG. 9 show perspective and top views similar to the view as shown in FIG. 6 but where the switch frame 80 has been omitted to reveal the first Gray code pattern 41 and the second Gray code pattern 51.

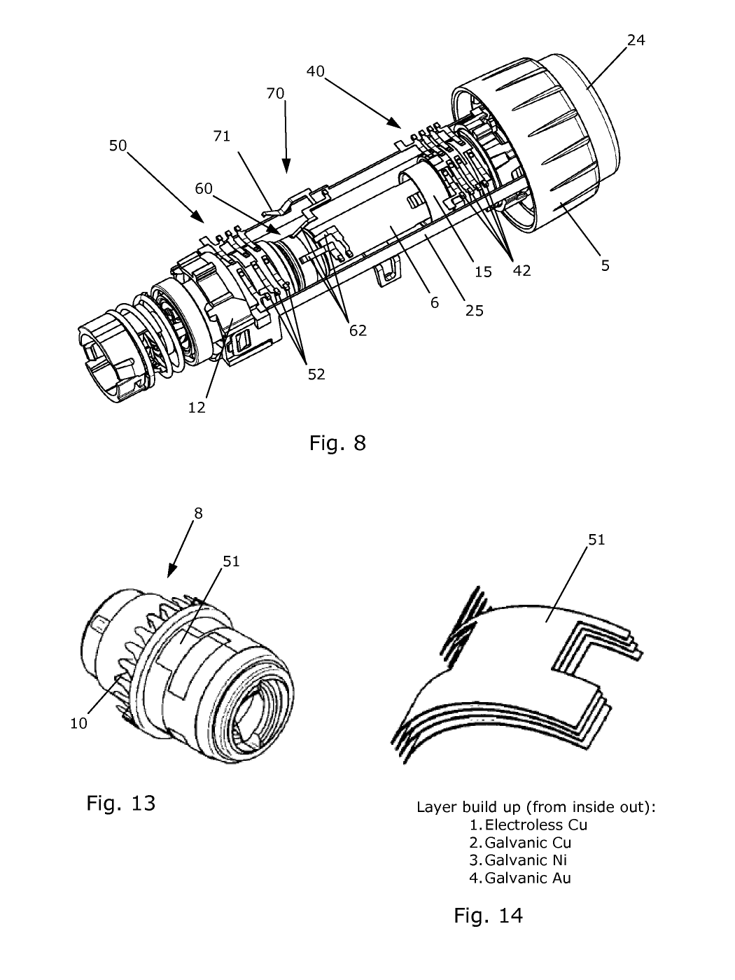

Further, FIG. 8 and FIG. 10 show similar perspective and top views where the contact arms of the switch frame 80 are visible.

The first Gray code pattern 41 is schematically represented in FIG. 11a and the second Gray code pattern 51 is schematically represented in FIG. 11b. The Gray code patterns 41 and 51 are based on a click mechanism associated with the counter-element 15 wherein 24 rotational steps are provided for each full revolution of dose knob 5 relative to the housing component 2. In this embodiment, the Gray code patterns have a rotational resolution corresponding to the dose increments defined by the click-mechanism, i.e. a pattern which changes state every 15 deg. angle of rotation. In other embodiments, the resolution of the Gray code patterns may be provided as two or three times the resolution defined by the click mechanism.

The first and second Gray code patterns have a code length of 8 codes (Index 0 through Index 7) and are each disposed within a 120 deg. span pr. sequence. Hence, for each full revolution that the counter-element 15 and locking nut 8 undergoes, the contact arms will swipe the respective Gray codes three times.

Each of the first and second Gray code patterns comprises separate tracks formed as a number of circular bands. A first circular band defines a continuous electrically conducting ground pattern (designated Ground SW). A set of two contact arms provides for redundant galvanic coupling to the first circular band of the Gray code patterns. The Gray code patterns further comprises two circular patterned bands each defining generally isolating fields of angular width 75 deg. spaced apart by 45 deg. conductive traces. The first of the two circular patterned bands is offset by an angle of 15 deg relative to the other of the two circular patterned bands. Contact arms designated SW 1, SW 2 are arranged to cooperate with the first circular patterned bands and contact arms designated SW 3 and SW 4 are arranged to cooperate with the other. The contact arms SW 1 and SW 2 are positioned 30 deg. apart. Also the contact arms SW 3 and SW 4 are positioned 30 deg. apart.

The first Gray code pattern further includes a further track forming a circular band of alternating conducting and isolating fields each having an angular width of 15 deg. This circular band is provided as a wake-up track. Also for this track a set of two contact arms spaced 30 deg. apart swipes this circular band and provides for redundant electrical connection.

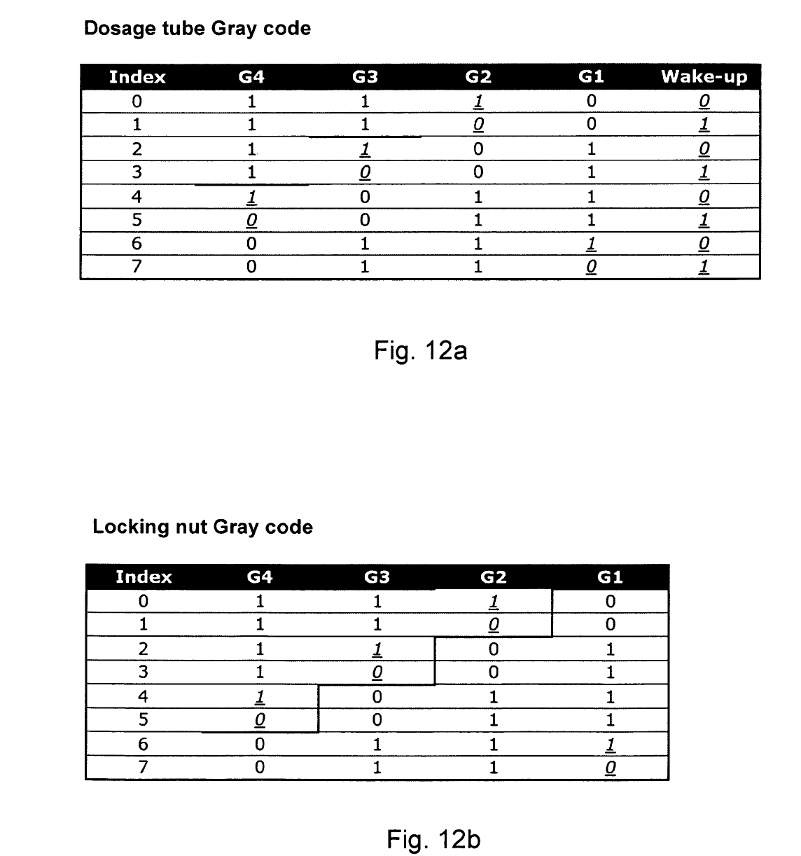

FIGS. 12a and 12b show table values of the first and the second sensor arrangement for each of the sequences Index 0 through Index 7 for a Gray code lay-out as shown in FIGS. 11a and 11b respectively. Both Gray code patterns provide an absolute measure of the rotational position within a sequence of 8 rotational positions.

As noted above, a switch that opens will not generate a rising voltage on its corresponding input since the pull-up resistor for the input has been disabled. Hence, having a Gray code pattern as defined in FIGS. 11b and 12b only the transitions 0-1, 2-3, 4-5 and 6-7 can be detected when rotating that particular Gray code pattern clockwise, and 0-7, 2-1, 4-3 and 6-5 can be detected when rotating counter-clockwise. Hence, by means of the additional wake-up track referred to above it is ensured that the pull-up resistors are enabled when needed. Referring to the table shown in FIG. 12a, for the first Gray code pattern 41 shown in FIG. 11a, it is noted that there will always be a switch that closes when going from an index to its neighbour index in either rotational direction. Hence a detectable level will always occur.

For the second Gray code pattern 51 which is associated with the locking nut 8 another implementation is chosen. Here all pull-up resistors are enabled whenever the fourth sensor arrangement 70 detects that the injection button 24 is pressed in; and deactivated when the fourth sensor arrangement 70 detects that the injection button is in its non-depressed state. In this way the second sensor arrangement 50 associated with the locking nut 8 and relating to dosing is only consuming power when the device 1 is actually in Dosing Mode.

As noted above, the third sensor arrangement 60 provides information as to whether the piston driver 6 is within a pre-defined axial distance from the position the piston driver 6 assumes at the end of dose position. In one form, the third sensor arrangement 60 may be based on a simple principle of two contact arms being connected by a conductive circular band 61 arranged fixedly relative to piston driver 6 wherein the conductive circular band is provided between adjacent regions of electrically insulating material. When the conductive circular band is not in the proximity of its end of dose position, i.e. further away than 0 to 7 index positions from the end of dose position the conductive circular band connect the two contact arms and consequently the switch will remain in an open state. When the piston driver 6 reaches a point in the proximity of its end of dose position (an arbitrary point that is within 0-7 index positions from the end of dose position), the cylinder will connect the switch arms and cause the third sensor arrangement 60 to enter a closed state.

In the shown embodiment, the third sensor arrangement 60 is provided as three contact arms 62 cooperating with conductive cylinder 61, providing two separate state changes occurring at two mutual offset axial positions of piston driver 6 relative to the locking member 8. Such configuration provides increased reliability in safely detecting whether or not the piston driver 6 is within close proximity to its end of dose position.

Electrically, the sensor is configured as a switch connected to ground, and the corresponding input to the electronic control circuit is held high by a pull-up resistor to ensure a well-defined signal level.

The fourth sensor arrangement 70 is based on a switch being closed. In the depicted embodiment a contact arm 72 is manipulated by a flange (not shown) on the connecting part 25. When the injection button is not activated (not pushed in) the flange will not activate the switch and consequently the switch will remain in an open state. When the injection button 24 is pushed in, the flange will perform an axial movement and cause the fourth sensor arrangement 70 to enter a closed state. Electrically the sensor is configured as a switch connected to ground, and the corresponding input to the electronic control circuit is held high by a pull-up resistor to ensure a well-defined signal level.

In other embodiments, the third sensor arrangement 60 and/or the fourth sensor arrangement 70 may include a similar manufacturing process using LDS as described above having a hard gold layer provided on the outer surface. For example, the conductive cylinder 61 may be disposed on a non-conductive thermoplastic support either integrally formed with piston driver 6 or fixedly attached to piston driver 6, where the above described metal layer distribution (see FIG. 14) is used.

The mechanical coupling between the piston driver 6 and the locking nut 8 during dose setting (dialing up and dialing down) as well as during dosing means that the first and second Gray code patterns 41 and 51 will always end up at the same relative rotational position after a complete dosing has taken place. Hence, in the end of dose state of the device 1, the index of the first Gray code pattern 41 will be the same as the index of the second Gray code pattern 51 provided that the two Gray code patterns during manufacture have been aligned corresponding to an alignment in the end of dose state of the device 1.

As noted above, the dose setting mechanism may be designed to cover a dosable range that may be chosen as 80 or 100 index positions. Due to this and due to the fact that the shown embodiment utilizes Gray code patterns which only provide an absolute detection of the rotational position within a sequence of 8 rotational positions (corresponding to 120 deg. of rotation) the monitoring during operation of the device 1 is based on counting the number of full sequences as well as fractional sequences of rotation performed during relative rotational movement between the piston driver 6 and the locking nut 8. Hence, there will be a multitude of relative rotational positions between piston driver 6 and locking nut 8 where the signals from the first and second sensor arrangements are the same. Likewise, there will be a number of relative rotational positions between piston driver 6 and locking nut 8 which correspond to the relative rotational position at the end of dose state of the device 1. Therefore, the synchronization between the first and the second sensor arrangements are being monitored.

In the above sensor configuration, the exact adjusted dose size and/or the total amount of an expelled dose will not be detectable when basing the monitoring solely on instantaneous data provided by the first sensor arrangement 40 and the second sensor arrangement 50. Should one or more interrupts be missed during operation of the device there will be a risk that the synchronization between the electronic sensor system and the mechanical system may fail.

In order to ensure synchronization between the mechanical system and the electronic system, the information provided by the third sensor arrangement 60 is utilized which provides a detection that the relative rotation between piston driver 6 and locking nut 8 is within 1 sequence (0-7 Index positions) from the end of dose state. Combining this information and the differential information from the first sensor arrangement 40 and the second sensor arrangement 50 a detection of the exact end of dose state is deductible. If a discrepancy should occur between the continuous monitoring and instantaneous information obtained from the sensor arrangements 40, 50, 60 and 70, the electronic control circuit will detect this as a failure and provide a warning indication to the user of the device. If the error is one of a recoverable kind, the device may be reset by means of the signals from the first sensor arrangement 40, the second sensor arrangement 50 and the third sensor arrangement 60 and the synchronization between the mechanical system and the electronic system may be recovered. If the error is irrecoverable, a warning as to this instance may be indicated to the user.

Due to the rotational stop surfaces of the piston driver 6 relative to the locking nut 8 at the end of dose state, the relative position are well defined and thus allows the device to reset itself during normal operation, e.g. during operation of the injection button 24 and/or the dose knob 5. Hence, the device may be so adapted that the first and the second sensor arrangements synchronizes automatically when the device is in the end of dose state.

The above described sensor values are used for estimating the dose as set and the dose as expelled so as to provide an indication on a display of the device (not shown). In the shown embodiment, during a dosing operation, the display may be configured to continuously show the part of a set dose that remains to be injected, e.g. as defined by the display refresh rate.

The electronic control circuit of the injection device 1 may further include a memory circuit adapted to hold information relating to a plurality of set and/or injected doses and the timing information relating to each such dose. Hereby the dosing history may be browsed through for example by utilizing the injection button 24 as a means for stepping through the injection history.

The electronic control circuit of the injection device 1 may in addition, or as an alternative, be provided with means for communicating the contents of the memory to an external apparatus, such as a personal computer, a mobile communication terminal such as a SmartPhone or such as a glucose meter (BGM/CGM). Such means for communication may be provided by means of an optical port such as an IR port, an RF communication antenna such as for communicating via Bluetooth or NFC, or via cable connection, etc.

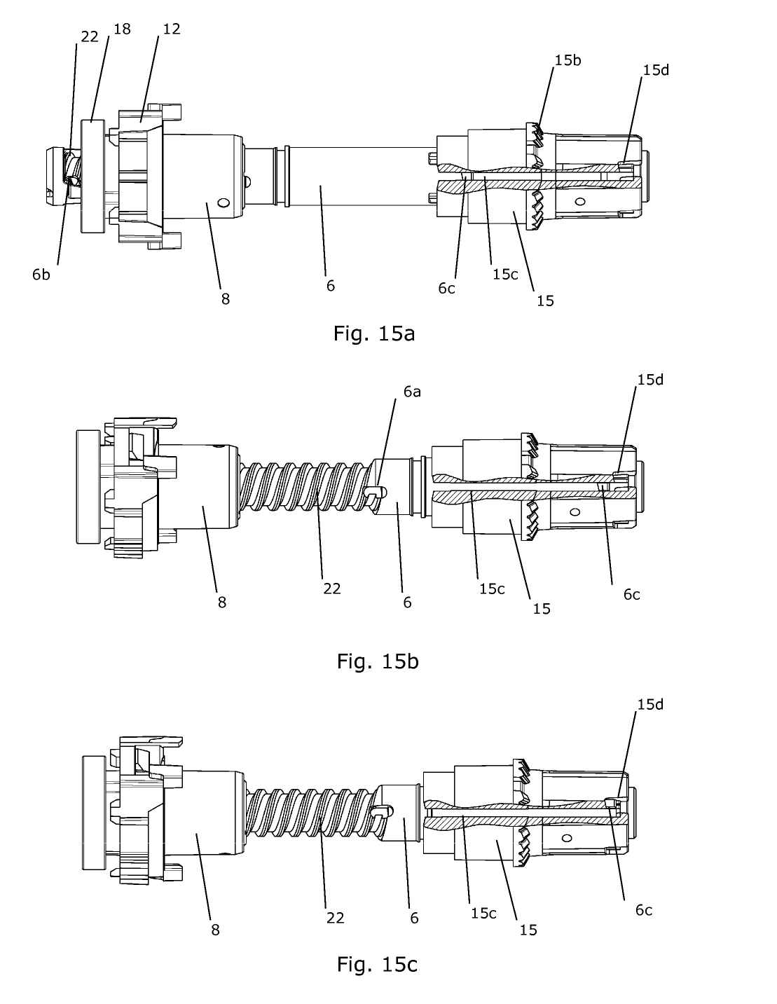

Now turning to FIGS. 15a-15c (all showing side views) and FIGS. 16a-16c (all showing cross sectional side views), these drawings depict selected components of an embodiment of an injection device 1 in first through sixth assembling states during manufacture of the injection device. In these drawings, the parts that are shown and that correspond to similar parts in the embodiments shown in FIGS. 1-5, 6-10 and 13-14 have been provided with identical reference numerals. The shown embodiment offers simplified manufacture of the device 1 wherein a subassembly including the piston driver 6, the spring device 19 and the counter-element 15 may be formed and arranged in an interlocked state where the spring device 19 is releasably retained in a pre-tensioned state. After forming the subassembly (as shown in FIGS. 15a-15c), the subassembly is easily inserted into the housing component 2 of the device 1 (shown in FIG. 16a) followed by further assembling steps (see FIGS. 16b-16c).

As shown in FIG. 15a, which depicts a side view with a partly sectioned counter-element 15, the piston driver 6 is mounted in the minimum dose setting position relative to the counter-element 15. Beforehand, the spring device 19 is inserted into the piston driver 6 and compressed between a proximally facing annular support surface formed internally in piston driver 6 and a distally facing support surface formed internally in counter-element 15. As the spring 19 is accommodated internally in piston driver 6 and counter-element 15, the spring device 19 is not viewable in the side views shown in FIGS. 15a-15c.

It is to be noted that FIG. 15a additionally shows the locking nut 8, the ball bearing 18 and the locking item 12 as forming the subassembly. Also the piston rod 7 may be assembled at this point but this has been omitted from the figures. Alternatively, these components may be included in the assembly after the state shown in FIG. 15c.

The piston driver 6 is provided with radially extending protrusions 6c positioned at the proximal end of the piston driver (see also FIG. 5). In the shown embodiment the number of protrusions 6c is four but other number of protrusions such as one, two, three or more protrusions may be selected as well. The protrusions 6c in the shown embodiment are equally spaced around the circumference of piston driver 6. Each protrusion 6c is adapted to be received in a corresponding longitudinal extending track 15c formed in counter-element 15 thereby forming sets of tracks and track followers. Hence, for relative axial positions between piston driver 6 and counter-element 15 between the minimum dose setting state (the zero dose setting shown in FIG. 15a) and the maximum dose setting state (shown in FIG. 15b), the piston driver 6 is configured to be rotationally locked to the counter-element 15 so that the piston driver 6 follows rotation of the counter-element 15.

As shown in FIG. 15c, for each of the longitudinal extending tracks 15c, at the proximal end thereof, a respective recess is formed to one side thereof in a particular circumferential direction. Each recess forms an interlock track 15d allowing a respective protrusion 6c of piston driver 6 to be received in the interlock track 15d. Hence, when the protrusions 6c align axially with the interlock tracks 15d, the piston driver 6 may be rotated slightly with respect to the counter-element 15 from a rotational position aligned with the longitudinal extending tracks 15c to an interlock position. In the shown embodiment of an injection device that is designed for a maximum dose setting limit of 100 index steps, the axial position of the interlock may be selected as a relative axial position between the piston driver 6 and the counter-element 15 corresponding to slightly more than 100 index steps, such as 102 index steps.

As apparent from FIG. 15c, the interlock tracks 15d and/or the protrusions 6c may be formed with ramp shaped engaging surfaces for ensuring that the interlocked state is maintained during the subsequent manufacturing steps. In the shown embodiment, the proximal facing surface of each interlock track 15d may be formed slightly inclined with respect to a tangential direction. In the shown embodiment, the distal facing surfaces of the protrusions 6c are also formed inclined with respect to a tangential direction. Hence, when the spring device 19 exerts a proximally directed force on the counter-element 15, and when each of the distal facing surfaces of the protrusions 6c engage with the proximal facing surface of the corresponding interlock track 15d, the assembly formed by the piston driver 6, the spring device 19 and the counter-element 15 will be maintained in the interlocked state where the relative axial position between piston driver 6 and counter-element 15 is fixed. However, as indicated above, the interlocked state of the subassembly shown in FIG. 15c is releasable.

As shown in FIG. 16a, due to the spring device 19 being axially compressed to a degree larger than the normal operating range of the dose setting arrangement, the subassembly including the piston driver 6, spring device 19 and counter-element 15 is somewhat shorter and hence may easily be inserted into the housing component 2.

Hereafter, as shown in FIG. 16b the interlock between piston driver 6 and counter-element 15 may be released by twisting the counter-element 15 relatively to piston driver 6. Hereby the protrusions 6c will align rotationally with the longitudinal extending tracks 15c and hence enabling axial movement between piston driver 6 and counter-element 15. As shown in FIG. 16b, using the maximum dose stop formed by geometries 6b/8b, the counter-element 15 will move slightly in the proximal direction until it engages a bearing surface formed in the housing component 2. Hereafter, the piston driver 6 may be returned to the minimum dose setting shown in FIG. 16c, either by turning the counter-element 15 or by releasing the lock nut 8 allowing the tensed spring device 19 to force forward the piston driver 6. Hereafter, the remaining parts of the device may be incorporated into the assembly,

It is to be noted that the dose setting and injection mechanisms described above only relate to a few particular embodiments according to the invention. In accordance with the design aspects described above, other drive mechanisms such as the drive devices disclosed in US 2007/0088290 A1 may be utilized in accordance with the present invention.

1. A drive mechanism for an injection device configured for setting and expelling set doses of a drug from a drug-filled cartridge, the drive mechanism comprising:
a piston driver configured for rotation around and axis and for axially driving forward a piston accommodated in a cartridge, the piston driver comprising a piston driver thread that engages a thread of a further component of the injection device, wherein the piston driver during dose setting rotates and moves in an axial direction,
a piston rod coupling movement of the piston driver with movements of the piston during expelling of a set dose, the piston rod and the piston driver being coupled so as to enable telescopically lengthening of the piston rod and the piston driver during dose setting,
a rotatable counter-element arranged coaxially with the piston driver, the piston driver and the counter-element being configured for relative axial movement during dose setting from a minimum dose state to a maximum dose state, and
a spring device arranged between the piston driver and the counter-element, the spring device being increasingly tensioned when the dose setting is changed from the minimum dose setting state to the maximum dose setting state, said tension being releasable for driving forward the piston during dose expelling,
wherein one of the piston driver and the counter-element defines a longitudinal track extending along said axis and the other of the piston driver and the counter-element defines a track follower adapted to engage the longitudinal track, wherein the longitudinal track and the track follower controls the relative rotational position between the piston driver and the counter-element for relative axial positions between the piston driver and the counter-element from the minimum dose setting state to the maximum dose state, and
wherein a releasable interlock is provided that, when activated, maintains the piston driver and the counter-element in an interlocked state where the relative axial position between the piston driver and the counter-element is fixed, and wherein the releasable interlock is so configured that activation and/or release of the interlock requires the spring device to be tensioned further than at the maximum dose setting state.

2. A drive mechanism as defined in claim 1, wherein the interlock is so configured that a relative rotational movement between the piston driver and the counter-element is required for activating and/or releasing the interlock.

3. A drive mechanism as defined in claim 1, wherein the interlock is so configured that when piston driver and the counter-element is maintained in the interlocked state, the tension of the spring device exceeds the tension of the spring device obtained at the maximum dose setting state.

4. A drive mechanism as defined in claim 1, wherein the interlock is so configured that a force originating from the spring device acts to maintain the interlock in the interlocked state.

5. A drive mechanism as defined in claim 1, wherein the longitudinal track is configured for rotationally locking the piston driver relative to the counter-element from the minimum dose setting state to the maximum dose setting state.

6. A drive mechanism as defined in claim 1, wherein the longitudinal track has a pitch relative to said axis so that the piston driver and the counter-element rotates relatively to each other when being shifted between the minimum dose setting state and the maximum dose setting state.

7. A drive mechanism as defined in claim 1, wherein the piston rod defines said further component and wherein said telescopically lengthening of the piston rod and the piston driver by way of the piston driver thread engaging a thread defined by the piston rod.

8. A drive mechanism as defined in claim 1, wherein said telescopically lengthening of the piston rod and the piston driver by way the of ratchet mechanism.

9. A drive mechanism as defined in claim 1, wherein the track follower forms part of the interlock.

10. A drive mechanism as defined in claim 9, wherein an interlock track connects with the longitudinal track so that the track follower is receivable in the interlock track, the interlock track extending substantially in a circumferential direction sideways relative to longitudinal track so that activation and/or release of the interlock requires a relative rotational movement between the piston driver and the counter-element forcing the track follower along the interlock track.

11. A drive mechanism as defined in claim 10, wherein the interlock track forms a ramp shaped surface for releasably maintaining the piston driver and the counter-element in the interlocked state.

12. A method of assembling an injection device incorporating a drive mechanism according to claim 1,
wherein the method of assembling the injection device comprises:
providing the spring device, the piston driver and the counter-element,
forming a drive mechanism subassembly by arranging the piston driver, the spring device and the counter-element relative to each other and operating the piston driver and the counter-element relatively to each other so that the spring device is tensioned,
moving the piston driver and the counter-element relatively to each other into a state where the tension of the spring device exceeds the tension of the spring device obtained when in the maximum dose setting state,
activating the releasable interlock for maintaining the piston driver and the counter-element in an interlocked state wherein the relative axial position between the piston driver and the counter-element is fixed and wherein the spring device is in a tensioned state.

13. A method of assembling an injection device as defined in claim 12, wherein the method comprises a further step of providing a housing comprising:
positioning the assembly relative to the housing, and
releasing the interlock thereby enabling the piston driver and the counter-element to be axially moveable relative to each other between the minimum dose setting state and the maximum dose setting state.

14. A method of assembling an injection device as defined in claim 1, wherein the method step a) comprises a step of providing the spring device, the piston driver and the counter-element such that:
one of the piston driver and the counter-element defines a longitudinal track extending along said axis, and
the other of the piston driver and the counter-element defines a track follower adapted to engage the longitudinal track, wherein the longitudinal track and the track follower controls the relative rotational position between the piston driver and the counter-element for relative axial positions between the piston driver and the counter-element between the minimum dose setting state and the maximum dose state, and
wherein an interlock track connects with the longitudinal track so that the track follower is receivable in the interlock track, the interlock track extending substantially in a circumferential direction sideways relative to longitudinal track so that activation and/or release of the interlock requires a relative rotational movement between the piston driver and the counter-element forcing the track follower along the interlock track.

15. A method of assembling an injection device as defined in claim 12, wherein the method further comprises:
operating the piston driver and the counter-element relative to each other for setting the dose between the minimum dose setting state and the maximum dose setting state or vice versa.
